(19) United States
(12) Patent Application Publication
(10) Pub. No.: US 2009/0032411 A1
(43) Pub. Date: Feb. 5, 2009

(54) METHOD AND SYSTEM FOR DETECTING PHARMACOLOGICALLY ACTIVE SUBSTANCES BY MEASURING MEMBRANE CURRENTS WITH EXTRACELLULAR SENSORS

(75) Inventors: Ingmar Peitz, Eindhoven (NL); Peter Fromherz, Munchen (DE)

Correspondence Address: ROTHWELL, FIGG, ERNST & MANBECK, P.C., 1425 K STREET, N.W., SUITE 800, WASHINGTON, DC, 20005, US

(73) Assignee: Max-Planck-Gesellschaft zur Foerderung Der Wissenschaften e.V., Muenchen (DE)

(21) Appl. No.: 12/278,986

(22) PCT Filed: Feb. 8, 2007
(86) PCT No.: PCT/EP2007/001087

§ 371 Date: Sep. 22, 2008

Related U.S. Application Data
(60) Provisional application No. 60/771,876, filed on Feb. 10, 2006.

(51) Int. Cl. C12Q 1/02; C12M 1/00; C12N 5/00; G01N 27/26; C12M 1/34

(52) U.S. Cl. 205/777.5; 435/325; 204/403.01

(57) ABSTRACT

The present invention relates to a bioelectronic device comprising a living cell which is in operative contact with an extracellular planar potential-sensitive electrode, e.g. a field effect transistor. The cell comprises first and second ion channel/receptor systems which are responsive to stimuli. The ion channels are selected such that the ion flux of the first ion channel is directed against the ion flux of the second ion channel. Thus, the device is suitable as a bioelectronic sensor. Further, the invention relates to a method for determining the response of the cell to a stimulus. The method is e.g. suitable for drug screening.

The present invention relates to a bioelectronic device comprising a living cell which is in operative contact with an extracellular planar potential-sensitive electrode, e.g. a field effect transistor. The cell comprises first and second ion channel/receptor systems which are responsive to stimuli. The ion channels are selected such that the ion flux of the first ion channel is directed against the ion flux of the second ion channel. Thus, the device is suitable as a bioelectronic sensor. Further, the invention relates to a method for determining the response of the cell to a stimulus. The method is e.g. suitable for drug screening.

To determine the pharmaceutical effect of test substances, often so-called cellular screening assays are performed in which a cell to be tested containing a receptor system is brought into contact with a test substance in order to examine its function as an effector on the cellular receptor system.

U.S. Pat. No. 6,602,399 discloses bioelectronic devices which combine receptor-effector systems with the functional characteristics of ion channels. The activity of these ion channels is modulated due to the effect of the receptor-effector system. This modulation can be detected by an extracellular planar potential sensitive electrode.

There is, however, still a need for devices and methods which allow a more efficient screening of many cells. This could simplify the procedure of pharmaceutical tests.

Thus, a subject matter of the present invention is bioelectronic device comprising
a) a cell which expresses (i) a first ion channel/receptor system wherein said first ion channel is responsive to a change in the characteristics of the first receptor and (ii) a second ion channel/receptor system wherein said second ion channel is responsive to a change in the characteristics of the second receptor,
   wherein the ion flux of the first ion channel is directed against the ion flux of the second ion channel, and
b) an extracellular planar potential-sensitive electrode wherein the cell is in operative contact with said electrode.

The device of the present invention comprises a living cell. This cell may be a microorganism, e.g. a bacterial cell or a yeast or fungal cell. Preferably, however, the cell is a eukaryotic cell, more preferably, a mammalian cell. Further, it is preferred that the cell overexpresses the ion channel/receptor systems, i.e. the cell is manipulated, e.g. by genetic engineering or mutation in a way that components of the ion channel/receptor systems are expressed in a higher amount than in a comparative untreated cell. More preferably, the cell is transfected, e.g. stably transfected with nucleic acid molecules encoding components of the ion channel/receptor systems. In this embodiment of the invention the cell comprises heterologous nucleic molecules which encode at least a part of the components of the ion channel/receptor systems and which allow overexpression of said components.

The device of the present invention expresses at least a first ion channel/receptor system and at least a second ion channel/receptor system. By selecting the ion channels such that the ion flux of the first ion channel is directed against the ion flux of the second ion channel, a depolarization/repolarization of the cell membrane is effected resulting in an increased signal in response to a change in the characteristics of at least one receptor associated with one of the ion channels can be measured.

Preferably, the first ion channel and the second ion channel are selected such that they direct a countercurrent flux of ions with a same charge, e.g. wherein the first ion channel directs an ion flux of a first ion species into the cell and the second ion channel directs an ion flux of a second species out of the cell, wherein the first and the second ion species have the same charge, i.e. both ion species have a positive charge or both ion species have a negative charge. The ions may be cations, e.g. sodium and/or potassium ions. Alternatively, the ions may be anions, e.g. chloride ions.

In a further embodiment, the first ion channel and the second ion channel are selected such that they direct a co-current flux of ions with a different charge, e.g. wherein the first ion channel directs an ion flux of a first ion species into the cell and the second ion channel directs an ion flux of a second ion species into the cell, wherein the first and the second ion species have a different charge, i.e. one species has a positive charge and the other species has a negative charge. In this embodiment, a cation species, e.g. sodium and/or potassium ions, may be combined with an anion species, e.g. chloride ions.

An ion channel/receptor system comprises a polypeptide or a plurality of polypeptides. On the one hand, an ion channel/receptor system comprises an ion channel component, e.g. a polypeptide or a plurality of polypeptides being capable of mediating an ion, i.e. cation and/or anion current through a cell membrane. On the other hand, an ion channel/receptor system comprises a receptor component which is responsive to stimuli. The receptor may be the ion channel or a part of the ion channel. The receptor, however, may be a molecule which is different from the ion channel, which is, however, in operative connection with the ion channel, e.g. a change in the functional and/or conformational state of the receptor results in a change of the functional state of the ion channel thus resulting in a detectable change of ion current through the cell membrane. The stimuli by which the receptor may be mediated are preferably selected from changes in the potential (inside or outside the cell), the presence or absence of effectors, e.g. ligands of the receptor, illumination, mechanical stimulation, stimulation by stimulation spots on the electrode or combinations thereof. For example, the binding of ligands to a receptor may cause the production of second messenger molecules which interact with the ion channel.

Preferred examples of ion channels are extracellular ligand-gated channels, e.g. serotonin receptors, such as 5-HT3, e.g 5-HT3A, nACh receptors, GABA_{A} receptors, glycine receptors, P2X receptors, NMDA receptors, AMPA receptors and kainate receptors; intracellular ligand-gated channels like InsP₃-gated channels, CNG channels and DAG-gated channels, e.g. coupled to a heterologous receptor system, and voltage-gated potassium channels like Kv1.1, Kv1.2, Kv1.3, Kv1.4 and all other Kv channels; and voltage-gated chloride channels such as ClC0, ClC1, ClC3. Preferred examples of receptor systems coupled to ion channels are G protein-coupled receptors (GPCR), receptor tyrosine kinases and T-cell receptors. Further receptor systems are metabotropic neurotransmitter receptors for serotonin, glutamate, acetylcholine and/or GABA. These receptor systems may be responsive to an extracellular ligand and produce a second messenger, which may act as a ligand to an intracellular ligand-gated ion channel.

The cell is cultivated on a planar potential-sensitive electrode. Methods of cultivating cells on planar potential-sensitive electrodes are disclosed e.g. in S. Vassanelli, P. Fromherz “Neurons from Rat Brain Coupled to Transistors” Appl. Phys. A 65, 85-88 (1997). By means of these cultivation cells are obtained, which grow on the potential-sensitive regions of the electrode resulting in an operative contact of the cell and the electrode.

When a cell is attached to the electrode surface, which may be oxidized silicon, other insulated semiconductors or metal, the cell membrane and the electrode surface are separated by a cleft which may be filled with an electrolyte. The electrode is preferably electrically insulated against the culture medium of the cell. Thus, a sandwich structure is formed of e.g. silicon, silicon dioxide, cleft, cell membrane and cell interior. The width of the cleft is usually in the range of about 10 to about 100 nm, e.g. about 50 nm.

The electrode may be integrated on, e.g. embedded in a chip. The chip may comprise further devices such as stimulating spots, transistors etc. Preferably the chip has at least one integrated field-effect transistor comprising at least one source and drain or an electrode as stimulating spot for applying voltages. The potential sensitive electrode, however, may also be a metal electrode which may be integrated on a chip. The chip may comprise a plurality of electrodes, e.g. field-effect transistors, for example at least 10, preferably at least 100, and more preferably, at least 1,000 electrodes on a single chip.

In the device of the present invention, the cell preferably has an integral membrane structure, i.e. there is no electrode, e.g. patch clamp, inserted into the membrane of the cell. This structural integrity leads to an increase in stability of the system, particularly allowing a plurality of identical or different measurement cycles without destroying the cells.

The bioelectronic device may comprise a single cell or a plurality of cells each in operative contact with an electrode. For example, the device may comprise at least 10, and preferably at least 100 cells on a single chip. The cells may be identical, i.e. they may contain identical first and second ion channel/receptor systems, or they may be different, i.e. they may contain different combinations of ion channel/receptor systems.

The functional characteristics of the first and second ion channels in the cell may include an opening of the channels in response to stimuli which will cause an ion current or flux to flow through participating channels. These ion currents will also flow in the region of operative contact between cell and electrode resulting in a detectable signal which can be measured. The detectable signal may be e.g. a voltage drop due to a junction resistance by the narrow cleft between cell and substrate or the change of the surface potential of the electrode due to diffuse ion concentration changes in the operative contact zone.

A change in functional characteristics e.g. conductivity of the ion channel changes the ion current and therefore the electrical signal detected by the electrode. Since the ion channels are responsive to the effector-receptor system, an alteration in the effector-receptor system will modulate the opening of the ion channels and thus result in a detectable signal.

Ion channels, particularly the gating characteristics thereof, can be modulated by different methods, e.g. by voltage modulation across the membrane (voltage-gated ion channels), by ligands acting on the intracellular and/or extracellular side of the channel (ligand-gated ion channels), by mechanical changes (mechanically-gated ion channels) or by combinations thereof.

Voltage-gated ion channels, i.e. ion channels which are voltage sensitive, will change their conductivity with the potential drop over the membrane (Vₘ=Vᵢₙₜᵣₐ−Vₑₓₜᵣₐ). If the electrolyte, i.e. the culture medium in which the cell is grown, is grounded (Vₑₓₜᵣₐ=0 mV) this potential drop equals the intracellular membrane (Vₘ=Vᵢₙₜᵣₐ). This potential drop may be measured. In another embodiment, the conductivity of voltage-gated ion channels may be changed by voltage modulation due to an interaction with other ion channels, e.g. by means of an action potential. Vₘ is changed due to ion currents flowing into a cell through different ion channels. This co-operation of several ion channels influences the potential drop over the membrane leading in some cases to an action potential. Moreover, the potential difference between intracellular and extracellular side of the membrane may be modulated by using stimulation spots on the electrode.

A stimulation spot may be integrated next to the potential-sensitive electrode being in operative contact to the cell (Stett et al., Phys. Rev. E 55 (1997), 85). Thus, a device with the features of stimulation and recording may be built. A stimulation spot can, e.g. trigger an action potential which then will be recorded by the extracellular electrode.

Ligands can modulate ion channels preferably by two mechanisms, ionotropic and second messenger systems. In an ionotropic system the ligand molecules bind directly to the ion channels and alter their gating characteristics, e.g. intracellular Ca²⁺ shifts the gating curve of some K⁺ channels (DiChiara and Reinhard, J. Physiol. 489.2 (1995), 403). In second messenger systems the ligand molecules bind to a receptor which will first trigger some other molecules before the ion channel is influenced, e.g. many glutamate-second messenger systems.

According to the present invention, two ion channels are combined wherein the ion flux of the first ion channel is directed against the ion flux of the second ion channel. In a preferred embodiment, one of the ion channels is a ligand-gated ion channel, e.g. an extracellular ligand-gated ion channel or an intracellular ligand-gated channel optionally coupled to a heterologous receptor system, and the other ion channel is a non-ligand-gated ion channel, preferably a voltage-gated ion channel. Preferred examples of extracellular ligand-gated ion channels are 5-HT3A, a cation channel which directs a cation flux into the cell or other extracellular ligand-gated channels, e.g. as described above. Preferred examples of voltage-gated ion channels are Kv1.3, a potassium ion channel which directs a potassium flux out of the cell, further voltage-gated potassium channels which direct a potassium flux out of the cell or chloride channels, which direct a chloride flux into the cell.

Surprisingly it was found that an effective signal may be generated in a cell comprising two ion channels with ion fluxes directed against each other. Without wishing to be bound by theory, the inventors believe that the signal is generated by a depolarization/repolarization process resulting from the combined activity of both ion channels together with differences in opening times for an ion channel, e.g. a ligand-gated ion channel in the part of the cell membrane in contact with the electrode as compared to the part of the cell membrane not in contact with the electrode.

The signal is preferably a voltage signal, particularly a change in the junction voltage in the cleft between the cell and the electrode. The signal may be generated by inducing a response to one or both of the ion channel-receptor systems in the cell. A response may be generated, e.g. by adding an agonist (or a test compound assumed to be an agonist) of a first ion channel/receptor system, e.g. comprising a ligand-gated ion channel. Alternatively, an antagonist or a presumed antagonist of a ligand-gated ion channel may be tested. In a further embodiment, agonists or antagonists of voltage-gated ion channels may be tested. The tests may be carried out optionally in the presence of further relevant compounds, e.g. antagonists or agonists of the second ion channel/receptor system.

Further, the present invention relates to a cell transfected with (i) at least one first nucleic acid molecule encoding components of a first ion channel/receptor system wherein said first ion channel is responsive to a change in the characteristics of the first receptor, and (ii) at least one second nucleic acid molecule encoding components of a second ion channel/receptor system wherein said second ion channel is responsive to a change in the characteristics of the second receptor, and wherein the ion flux of the first ion channel is directed against the ion flux of the second ion channel.

The cell is suitable as a component of a bioelectronic device as indicated above or as a component in any other device capable of determining ion currents, e.g. by using voltage-dependent dyes as disclosed in EP-A-1 553 396 which is herein incorporated by reference.

The cell and bioelectronic device of the invention are suitable as a sensor which allows the determination of a change in an environmental parameter as a detectable signal, e.g. on the electrode of the device, and which is suitable as a scientific tool for studying the conformational and functional states of membrane proteins.

Particularly, the environmental parameter is an effector for the receptor component of the first and/or second ion channel/receptor system. More particularly, the system is used to determine whether a test substance is capable of activating or inhibiting the receptor component of the first and/or second ion channel/receptor system. The receptor component may be a pharmaceutically relevant target molecule. Thus, the present invention provides a method for determining the response of a cell to a stimulus, comprising stimulating a bioelectronic device or a cell as described above, and determining the response to the stimulus, e.g. by contacting a test substance with the device or cell and determining a response of an ion channel/receptor system to the test substance. The response may be determined, for example, as an electric signal, e.g. a voltage signal, by the electrode of the bioelectronic device, or as an optical signal, e.g. by using a voltage-dependent dye.

In another embodiment, the bioelectronic device or the cell may be used as a sensor to determine the presence or the amount of a substance which acts as an effector to a receptor component in the cell.

Further, the invention shall be explained by the following figures and examples:

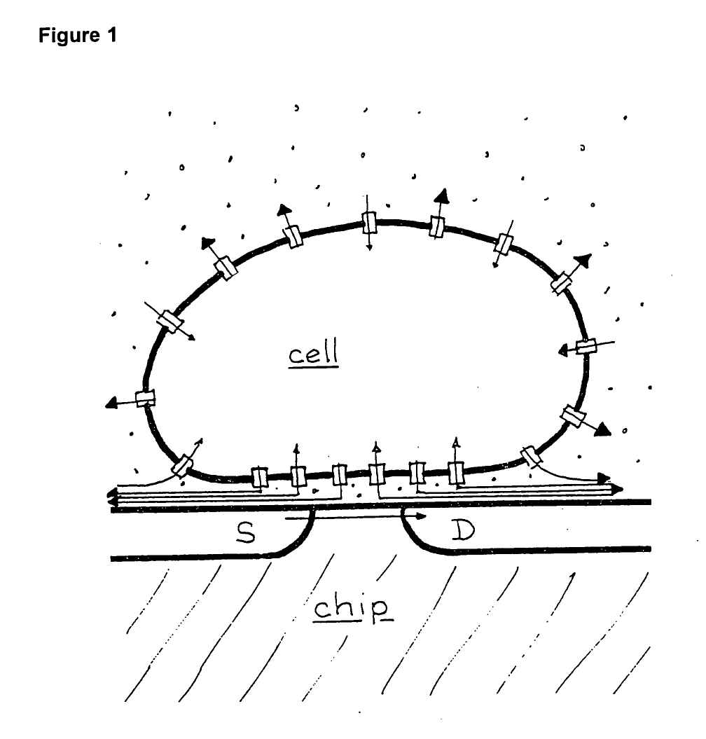

FIG. 1:

A schematic picture of an ionoelectronic sensor with a cell on an open field-effect transistor. The cell comprises first and second ion channel/receptor systems. The first ion channel/receptor system directs an ion current into the cell and the second ion channel/receptor system directs an ion current out of the cell. A thin cleft of electrolyte separates the attached membrane from the silicon dioxide of the silicon chip. A signal, e.g chemical signal, in the solution opens receptor channels in the free and in the attached membrane. Ionic current flows in both directions through the free and attached membrane, driven by a suitable thermodynamic force. The resulting superposition of ionic and capacitive current through the attached membrane flows along the narrow cleft and gives rise there to a voltage drop.

A measurable signal is generated by a depolarization/repolarization process resulting from the ion channel activity and by different opening times for channels in the narrow cleft between cell membrane and electrode compared to the channels in the free cell membrane. The resulting measurable change of extracellular voltage in the cleft plays the role of a gate voltage for the open field-effect transistor and modulates the electronic current from source (S) to drain (D) in the silicon chip.

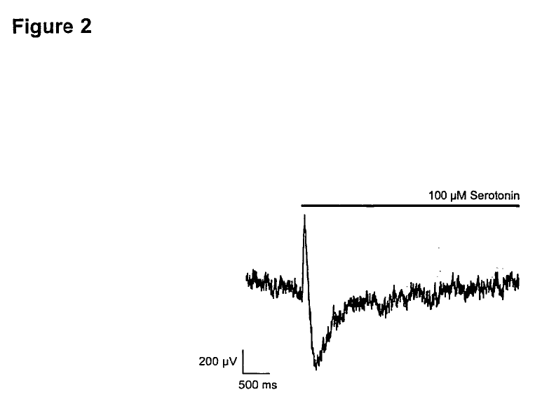

FIG. 2:

Recording of the extracellular voltage signal V_{J} in the cell-chip junction probed with a Field Effect Transistor gate. The cell was transfected with the ligand-gated ion channel 5-HT3A, and the voltage-gated ion channel Kv1.3 and stimulated with serotonin (100 μM). The bar on top of the image indicates the onset and duration of serotonin stimulation.

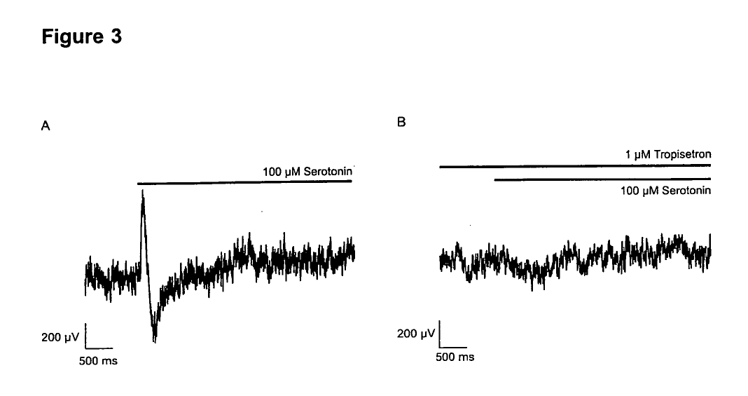

FIG. 3:

A Recording of the extracellular voltage signal caused by the application of 100 μM serotonin (bar on top) to another doubly transfected cell as recorded with a FET.

B Recording of the same cell during the application of tropisetron (first bar), a specific blocker of 5-HT3A. Tropisetron (1 μM) was present before and during the stimulation with serotonin (second bar). The signal was lost by inhibition of the current through the 5-HT3A channel.

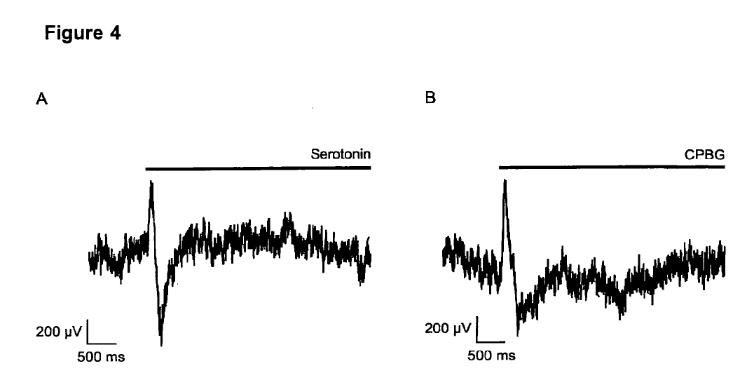

FIG. 4:

A FET recording of the extracellular voltage signal caused by the application of 100 μM serotonin (bar on top) to another doubly transfected cell.

B Recording of the extracellular voltage of the same cell during application with the agonist CPBG (100 μM, bar on top). Both agonists of the 5-HT3A receptor triggered a biphasic voltage signal.

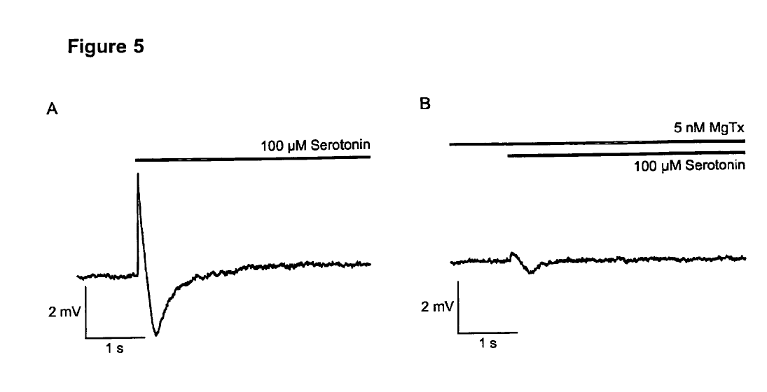

FIG. 5:

A Biphasic extracellular voltage signal of another doubly transfected cell, induced by the application of 100 μM serotonin (bar on top) and recorded with a FET under the cell.

B Recording of the same cell during the application of 100 μM serotonin (second bar) in the presence of 5 nM margatoxin (MgTx, first bar), a specific blocker of the Kv1.3-channel at this concentration. The signal was reduced by an incomplete inhibition of the current through the Kv1.3-channel.

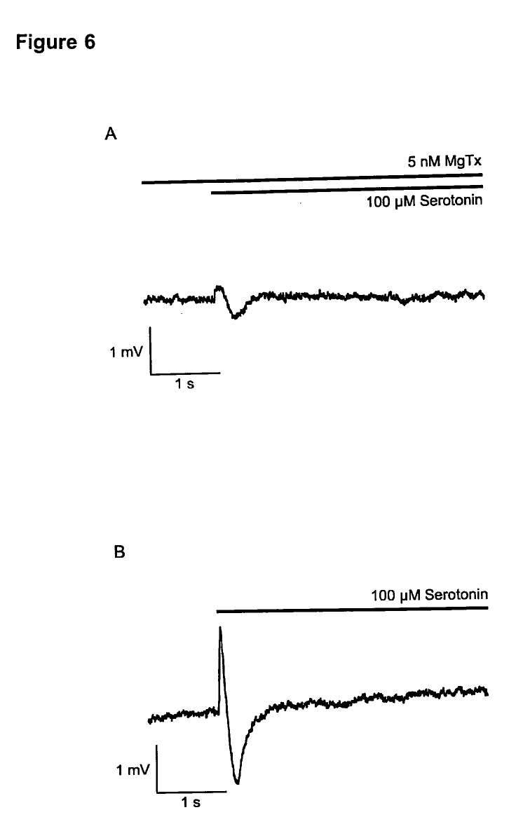

FIG. 6:

A FET recording of the extracellular voltage of a doubly transfected cell during serotonin stimulation (second bar) in the presence of 5 nM margatoxin (first bar).

B Signal of the same cell, triggered with serotonin (bar on top) after removal of margatoxin. A biphasic voltage signal was recovered.

### EXAMPLE

### 1. Materials and Methods

### 1.1 Cells and Plasmids

HEK293 cells were cultured in plastic dishes (Becton Dickinson, No. 353001), in Dulbecco's modified Eagle's medium (GIBCO, No. 21885-025), supplemented with 10% heat-inactivated fetal bovine serum. The cDNA of the human ligand-gated ion channel 5-HT3A was purchased from Invitrogen (Ultimate ORF clone collection, HORF01), and subcloned into the expression plasmid pcDNA3 (Invitrogen, No. V790-20), applying standard molecular biology methods. The construction of an expression plasmid for the voltage-gated potassium channel Kv1.3 was as described in J. Kupper: Functional expression of GFP-tagged Kv1.3 and Kv1.4 channels in HEK 293 cells. Eur. J. Neurosci. 10, 3908-12 (1998). The cells were stably transfected with one of the two expression plasmids using Geneticin (GIBCO, No. 11811-064), as the selection antibiotic (500 μg/ml). After generation of stable cell lines, the cells were transiently transfected with the other of the two expression plasmids together with the EGFP-C1 plasmid (BD Biosciences Clontech, No. 632317), which served as control for successful transfection. Transfections were done using the Effectene method (Qiagen, No. 301425), at 60-80% confluency.

### 1.2 Cell Culture on Silicon Chips

Silicon chips as described in M. Voelker, P. Fromherz: Signal transmission from individual mammalian nerve cell to field-effect transistor. Small 1, 206-210 (2005) with 128 Field Effect Transistors (FET) in two linear arrays and a culture chamber on top were cleaned, sterilized under UV-light and coated with 10 μg/ml fibronectin (Sigma, No. F2006). Cells were plated on these chips under normal culture conditions one day after transient transfection, and measurements were done one day after plating. Cells on transistor gates with the transiently expressed channels were identified by the green EGFP fluorescence.

### 1.3 Experimental Conditions and Solution Exchange

During experiments the cells were flushed permanently with standard extracellular solution, consisting of (in mM): KCl 5.4, NaCl 135, CaCl₂ 1.8, MgCl₂ 1 Glucose 10, Hepes 5 (pH adjusted to 7.4 with NaOH). The ligands serotonin, CPBG (agonists of 5-HT3A) tropisetron and margatoxin (antagonists of 5-HT3A and Kv1.3, respectively), were obtained from Sigma (No. H9523, C144, T104, M8278), and dissolved in extracellular solution.

Ligand solutions were applied rapidly (ms range), with a double barrelled glass pipette (Theta-Tube), connected to a piezoelectric actuator (Burleigh, LSS-3100). Different ligand solutions were directed to the glass pipette by an eight-channel valve control system (ALA, BPS-8).

### 2. Results

During application of ligands, the voltage in the cleft between transfected cells and the chip surface was probed with the gates of Field Effect Transistors under the cells. Changes in this junction voltage were due to the ionic currents through both of the ion channels in the cell membrane, as was proven in the following way:

Cells expressing only the ligand-gated ion channel 5-HT3A failed to cause a voltage signal in the cell-chip junction upon stimulation with serotonin. Cells expressing only the voltage-gated channel Kv1.3 likewise failed to produce a signal upon stimulation with serotonin. On the other hand, the agonists serotonin and CPBG of the serotonin receptor 5-HT3A both caused a biphasic voltage signal in the transistor when applied to cells expressing both channels. The specific antagonist tropisetron of the 5-HT3A receptor blocked the signal as does an antagonist of the Kv1.3-channel, such as margatoxin, which is a specific blocker of Kv1.3 in the selected concentration range. Therefore the system is sensitive to ligands of the 5-HT3A receptor as well as to blockers of the Kv1.3-channel. The biphasic nature of the signal is caused by depolarization and subsequent repolarization of the cell membrane as well as by two different opening times for the 5-HT3A channels in the adhered cell membrane as compared to the channels in the free cell membrane.

Thus we provided a bioelectric device to screen for ligands to ligand-gated ion channels and to voltage-gated ion channels in a non-invasive and fast manner which could easily be expanded to a plurality of cells being processed in parallel. This would yield a high throughput screening device. Furthermore, this system is also applicable for screening ligands targeting voltage-gated channels as it is also sensitive to these molecules.

1-30. (canceled)

31. A bioelectronic device comprising
(a) a cell which expresses (i) a first ion channel/receptor system wherein said first ion channel is responsive to a change in the characteristics of the first receptor and (ii) a second ion channel/receptor system wherein said second ion channel is responsive to a change in the characteristics of the second receptor,
wherein the ion flux of the first ion channel is directed against the ion flux of the second ion channel, and
(b) an extracellular planar potential-sensitive electrode wherein the cell is in operative contact with said electrode.
wherein the cell is transfected with a first nucleic acid molecule encoding components of the first ion channel/receptor system and with a second nucleic acid molecule encoding components of the second ion channel/receptor system.

32. The device of claim 31
wherein said cell is a eukaryotic cell.

33. The device of claim 31
wherein the cell overexpresses said first and/or second ion channel/receptor system.

34. The device of claim 31
wherein the cell is stably transfected, with nucleic acid molecules encoding components of said first and/or second ion channel/receptor system.

35. The device of claim 31
wherein said first and second ion channels are selected from voltage-gated ion channels, ligand-gated ion channels, mechanically-gated ion channels or combinations thereof.

36. The device of claim 35
wherein one of said first and second ion channels is a ligand-gated ion channel and the other of said first and second ion channels is a non-ligand-gated ion channel, preferably a voltage-gated ion channel.

37. The device of claims 35
wherein one of the first and second ion channels is an extracellular ligand-gated ion channel.

38. The device of claim 35 wherein one of the first and second ion channels is an intracellular ligand-gated ion channel optionally in combination with a heterologous receptor system, such as a G-protein coupled receptor (GPCR), a receptor tyrosine kinase or a T-cell receptor.

39. The device of claim 31
wherein the first ion channel and the second ion channel direct a flux of ion species with the same charge into the cell or out of the cell, respectively.

40. The device of claim 39
wherein the first ion channel and the second ion channel direct a flux of the same ion species into the cell or out of the cell, respectively.

41. The device of claim 39
wherein the ions are cations, e.g. potassium and/or sodium ions.

42. The device of claim 39
wherein the ions are anions, e.g. chloride ions.

43. The device of claim 31
wherein the first ion channel is a ligand-gated cation channel, which directs a cation flux into the cell or out of the cell.

44. The device of claim 37
wherein the first ion channel is selected from serotonin receptors such as 5-HT3, nACh receptors, GABA_{A} receptors, glycine receptors, P2X receptors, NMDA receptors, AMPA receptors, and kainate receptors.

45. The device of claim 38
wherein the first ion channel is selected from InsP₃ channels, CNG channels, and DAG-gated channels.

46. The device of claim 35
wherein the second ion channel is a voltage-gated potassium or chloride channel, which directs a potassium or chloride flux out of the cell or into the cell.

47. The device of claim 46
wherein the second ion channel is selected from Kv channels and CIC channels.

48. The device of claim 31
wherein the cell has an integral membrane structure.

49. The device of claim 31
wherein the electrode is located on a chip.

50. The device of claim 31
wherein the electrode is electrically insulated against the culture medium of the cell.

51. The device of claim 31
which comprises a plurality of electrodes, e.g. at least 10, preferably at least 100 and more preferably at least 1,000 electrodes on a single chip.

52. The device of claim 31,
which comprises a plurality of cells, which may be identical or different.

53. A cell transfected with (i) a first nucleic acid molecule encoding components of a first ion channel/receptor system, wherein said first ion channel is responsive to a change in the characteristics of the first receptor, and (ii) a second nucleic acid molecule encoding components of a second ion channel/receptor system wherein said second ion channel is responsive to a change in the characteristics of the second receptor, and
wherein the ion flux of the first ion channel is directed against the ion flux of the second ion channel.

54. Use of a bioelectronic device according to claim 31 as a sensor.

55. The use of claim 54 wherein a change in an environmental parameter is sensed as a detectable electrical or optical signal.

56. The use of claim 55 wherein the environmental parameter is an effector for the receptor component of an ion channel/receptor system.

57. Use of a bioelectronic device according to claim 31 in a drug screening procedure.

58. The use of claim 57 for the determination whether a test substance is capable of activating or inhibiting a receptor component.

59. A method of determining the response of a cell to a stimulus comprising stimulating a device according to claim 31, and determining the response to the stimulus.

60. The method of claim 59 comprising contacting a test substance with the bioelectronic device or the cell and determining the response of an ion channel/receptor system to the test substance.

61. Use of a cell according to claim 53 as a sensor.

62. Use of a cell according to claim 53 in a drug screening procedure.

63. A method of determining the response of a cell to a stimulus comprising stimulating a cell of claim 53, and determining the response to the stimulus.
